(19) United States
(12) Patent Application Publication
(10) Pub. No.: US 2021/0350922 A1
(43) Pub. Date: Nov. 11, 2021

(54) METHODS FOR TRACKING HYGIENE COMPLIANCE

(71) Applicant: GOJO Industries, Inc., Akron, OH (US)

(72) Inventor: Martin Joseph O'Toole, Chagrin Falls, OH (US)

(21) Appl. No.: 17/381,416

(22) Filed: Jul. 21, 2021

Related U.S. Application Data
(63) Continuation of application No. 15/200,314, filed on Jul. 1, 2016.
(60) Provisional application No. 62/187,968, filed on Jul. 2, 2015.

(51) Int. Cl. G16H 40/63; G16H 50/30; G16H 20/30; G16H 40/20; G16H 40/67

(52) U.S. Cl. CPC G16H 40/63; G16H 50/30; G16H 40/67; G16H 40/20; G16H 20/30

(57) ABSTRACT

Methods for creating personalized hand hygiene compliance protocols and for tracking hand hygiene compliance is disclosed herein. An exemplary method for creating personalized hand hygiene compliance protocols and for tracking hand hygiene compliance includes providing a plurality of dispensers, wherein the dispensers contain one of soap or sanitizer. The methodology further includes retrieving patient profile information from a patient profile database that relates to a patient and creating a personalized hand-hygiene compliance protocol for the patient as a function of the patient profile information. The hand-hygiene compliance protocol includes at least two conditions that require the patient to wash her hands with soap or sanitizer. The methodology further includes determining if a dispense from one of the plurality of dispensers was caused by the patient, logging the dispense from one of the plurality of dispensers and associating the dispense to the patient if the dispense was caused by the patient.

### REFERENCE TO RELATED APPLICATIONS

This application is a continuation application of U.S. patent application Ser. No. 15/200,314, which was filed on Jul. 1, 2016, and which is incorporated by reference in its entirety herein. In addition, this application claims priority to U.S. Provisional Patent Application Ser. No. 62/187,968, filed Jul. 2, 2015, the contents of which is incorporated herein by reference.

### TECHNICAL FIELD

A fluid dispenser is in communication with a remote monitoring system.

### BACKGROUND

Conventional fluid dispensers can communicate with a central computer to facilitate monitoring of various characteristics of the fluid dispenser.

### SUMMARY

In accordance with one embodiment, a method is provided for tracking hygiene compliance. The method comprises receiving a health condition of a patient and assigning, by a processor, a hygiene compliance protocol for the patient based upon the health condition of the patient. The hygiene compliance protocol defines suggested hygiene-related activities for the patient. The method further comprises monitoring the hygiene-related activities performed by the patient and comparing the hygiene-related activities performed by the patient to the suggested hygiene-related activities of the hygiene compliance protocol. The method further comprises logging, to an electronic profile, the suggested hygiene-related activities of the hygiene compliance protocol that were not performed by the patient.

In accordance with another embodiment, a method is provided for tracking hygiene compliance. The method comprises receiving a health condition of a patient and assigning, by a processor, a hygiene compliance protocol for the patient based upon the health condition of the patient. The hygiene compliance protocol defines suggested scheduled hygiene-related activities for the patient. The method further comprises monitoring the patient's non-hygiene related activity and adding a suggested behavior-based hygiene-related activity for the patient to the hygiene compliance protocol based upon the patient's non-hygiene related activity. The method further comprises monitoring the hygiene-related activities performed by the patient and comparing the hygiene-related activities performed by the patient to the hygiene compliance protocol and logging, to an electronic profile, the suggested scheduled hygiene-related activities of the hygiene compliance protocol that were not performed by the patient. The method still further comprises logging, to the electronic profile, the suggested behavior-based hygiene-related activities of the hygiene compliance protocol that were not performed by the patient.

In accordance with yet another embodiment, a method for tracking hand-hygiene compliance is provided. The method comprises receiving a condition of a patient and assigning, by a processor, a hygiene compliance protocol for the patient based upon the condition of the patient. The hand-hygiene compliance protocol defines suggested hand-hygiene related activities for the patient. The method further comprises receiving an RFID signal from the patient at a fluid dispenser and identifying the patient based upon the RFID signal. The method still further comprises comparing the actuation of the fluid dispenser by the patient to the suggested hand-hygiene related activities of the hygiene compliance protocol and logging, to an electronic profile, the suggested hygiene-related activities of the hygiene compliance protocol that were not performed by the patient.

### BRIEF DESCRIPTION OF THE DRAWINGS

Various embodiments will become better understood with regard to the following description, appended claims and accompanying drawings wherein:

FIG. 1 is perspective view depicting a fluid dispenser, with a lid of the fluid dispenser shown in a closed position;

FIG. 2 is a front perspective view depicting the fluid dispenser of FIG. 1, but with the lid of the fluid dispenser being shown in an open position;

FIG. 3 is schematic view depicting the fluid dispenser in association with an RFID tag and a central monitoring system;

FIG. 4 is an environmental view depicting a health care facility;

FIGS. 5A and 5B are schematic views depicting one example of a patient profile of the central monitoring system of FIG. 3;

FIGS. 6A and 6B are schematic views depicting another example of a patient profile of the central monitoring system of FIG. 3; and

FIGS. 7A and 7B are schematic views depicting yet another example of a patient profile of the central monitoring system of FIG. 3.

### DETAILED DESCRIPTION

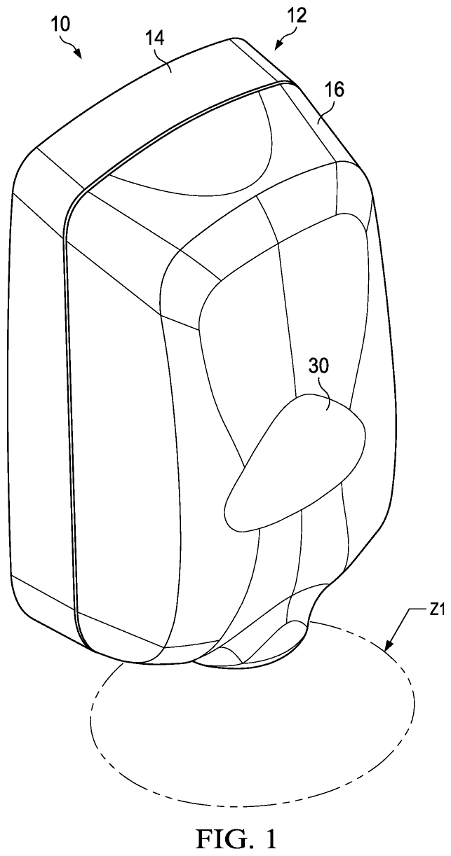

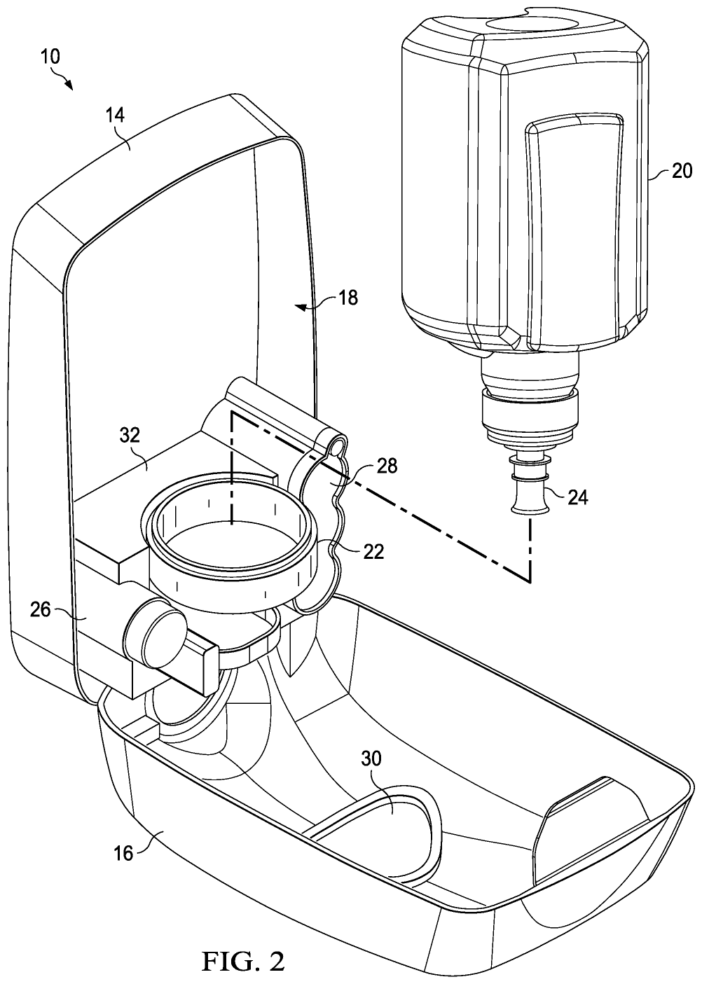

Embodiments are hereinafter described in detail in connection with the views of FIGS. 1-4, 5A, 5B, 6A, 6B, 7A, and 7B, wherein like numbers indicate the same or corresponding elements throughout the views. FIGS. 1-2 illustrate a fluid dispenser 10 which can dispense a variety of different types of fluids or liquids, such as, for example, soap, sanitizer, soil removing cleaner, lotion, shampoo, or conditioner, to the hands of a user. The fluid dispenser 10 can include a housing 12 that includes a base 14 and lid 16. The base 14 and the lid 16 can cooperate to define an interior chamber 18 which can house various components of the fluid dispenser 10, and can be configured to receive a fluid container 20. The fluid container 20 can contain a fluid or liquid to be dispensed from the fluid dispenser 10, onto the hands of a user. The lid 16 can be pivotable with respect to the base 14, as illustrated in FIG. 2, to reveal the interior chamber 18 to allow for replacement of the fluid container 20.

Referring now to FIG. 2, the fluid dispenser 10 can include a collar 22 that can receive a dispensation port 24 of the fluid container 20. The fluid dispenser 10 can also include a motor 26 and batteries 28 for powering the motor 26. With the fluid container 20 installed on the collar 22, the motor 26 can actuate a pump (not shown) to facilitate dispensation of fluid onto a user's hands below. The fluid dispenser 10 can include a proximity sensor (not shown) or other detection device that defines a detection zone Z1 (FIG. 1). A user can indicate a request for actuation of the fluid dispenser 10 by placing his/her hands (or other object) within the detection zone Z1, which can initiate operation of the motor 26 to dispense fluid onto the user's hands. In an alternative embodiment, a user can request actuation of the fluid dispenser 10 by manually actuating a pull bar (not shown) that facilitates dispensation of fluid onto the user's hands.

As shown in FIGS. 1 and 2, the lid 16 can define a viewing window 30, which can facilitate viewing into the interior chamber 18 defined by the base 14 and lid 16, for example, to determine whether the fluid container 20 is disposed within the fluid dispenser 10 and/or to determine the fill level of the fluid container 20.

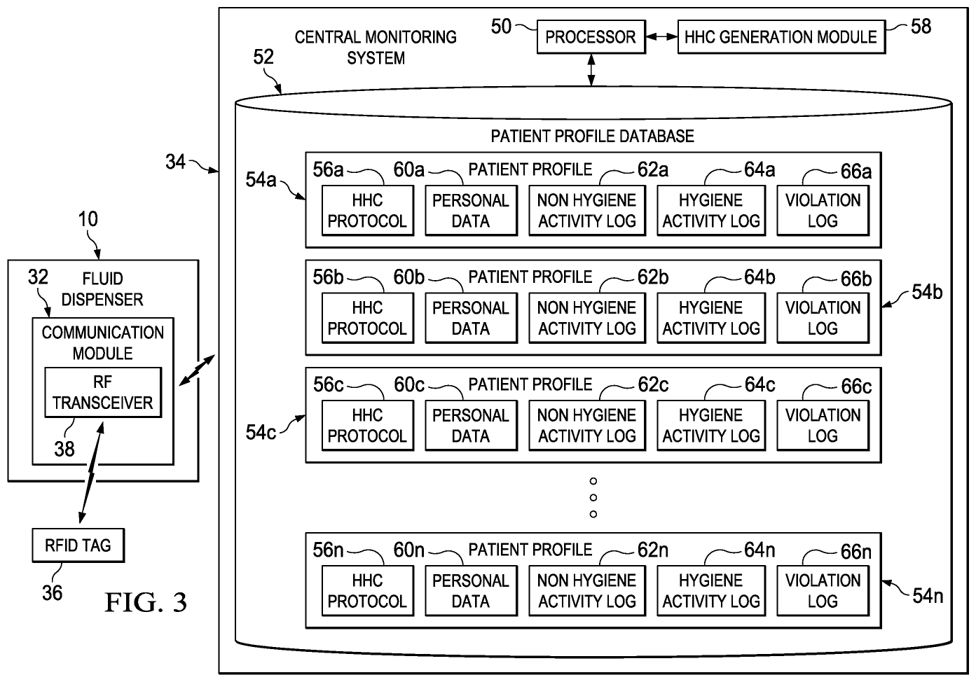

The fluid dispenser 10 can include a communication module 32, as illustrated in FIGS. 2 and 3. When the fluid dispenser 10 is installed in a facility, the communication module 32 can facilitate communication with a central monitoring system 34 of the facility. The central monitoring system 34 can be any of a variety of computing devices capable of monitoring electronic devices within the facility. While the fluid dispenser 10 and central monitoring system 34 can be installed in a variety of facilities (manufacturing facilities, food processing facilities, etc.), for purposes of explanation, the present disclosure is described in terms of a healthcare facility. As will be described in more detail below, when a patient uses the fluid dispenser 10 to sanitize his/her hands, the central monitoring system 34 can log the patient's use of the fluid dispenser 10. The central monitoring system 34 can also collect data from the fluid dispenser 10 to determine operational status of the fluid dispenser 10 (e.g., fluid level, dispensation count, powered on/off), maintenance alarms (e.g., refill needed, dispensation port clogged, inoperable alarm, powered off), or any of a variety of other suitable information about the fluid dispenser 10.

The communication module 32 and the central monitoring system 34 can include network and communication interfaces, either wired or wireless, that allow communication with each other across a network. The network and communication interfaces can be an Ethernet interface, a radio interface, a telephony interface, a Universal Serial Bus (USB) interface, or any other suitable communications interface. Example communication interfaces can include wired data transmission links such as Ethernet and TCP/IP, as well as PSTN communications links such as T1s (or better), integrated services digital network (ISDN), Digital Subscriber Line (DSL), or dialup modems that implement, for example, the point-to-point protocol (PPP). The communication interface can include wireless protocols for interfacing with private or public networks. For example, the network and communication interfaces and protocols can include interfaces for communicating with private wireless networks such as a WiFi network, one of the IEEE 802.11x family of networks, or another suitable wireless network. The network and communication interfaces can include interfaces and protocols for communicating with public wireless networks, using for example wireless protocols used by cellular network providers, including Code Division Multiple Access (CDMA) and Global System for Mobile Communications (GSM).

In one embodiment, to facilitate logging of the patients' use of the fluid dispenser 10, the patients of the healthcare facility can wear, or otherwise be associated with, a radio frequency identification (RFID) tag 36 that stores (e.g., in a memory module) personal information about the patient such as, for example, name, gender, mailing address, a unique identifier (e.g., social security number), medical condition, insurance information, or any of a variety of other suitable information that would facilitate identification of the patient. The communication module 32 (or other communication device) of the fluid dispenser 10 can include a radio frequency (RF) transceiver 38 (e.g., an RFID reader) that facilitates communication with the RFID tag. The RF transceiver 38 can define a communication zone (not shown). When the RFID tag 36 is outside of the communication zone Z1, the fluid dispenser 10 and the RFID tag 36 are not in communication. However, once the RFID tag 36 enters the communication zone, the fluid dispenser 10 and the RFID tag 36 can automatically establish a communication link such that the fluid dispenser 10 and the RFID tag 36 are in communication with each other.

When a patient approaches the fluid dispenser 10 (i.e., to request dispensation of fluid) and enters the communication zone, the communication module 32 and the RFID tag 36 can establish a communication link. When the patient uses the fluid dispenser 10 (e.g., by placing his/her hands in the dispensation zone Z1 or actuating the pull bar), the central monitoring system 34 can log the patient's usage of the fluid dispenser 10 using the patient's personal information from the RFID tag 36. It is to be appreciated that a patient can be identified according to any of a variety of suitable alternative identification techniques. Thus while an RFID tag 36 and RF transceiver 38 are depicted in FIG. 3, any suitable contacting or non-contacting patient identification techniques can be utilized, such as QR codes and optical scanners, keypads, biometric scanners, and the like.

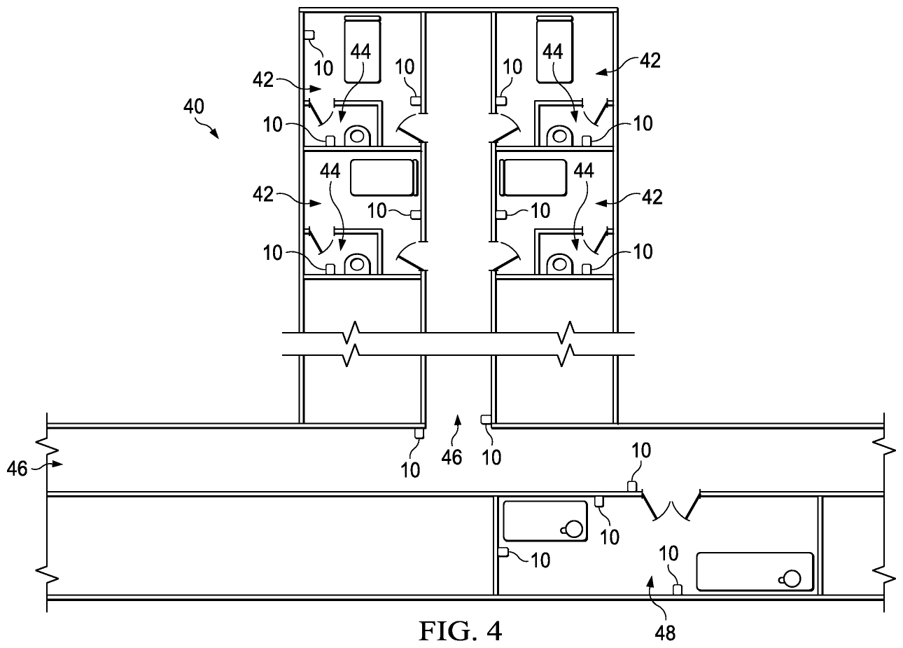

An example layout of a health care facility 40 is illustrated in FIG. 4. The health care facility 40 is shown to include a plurality of fluid dispensers 10 provided throughout the health care facility 40 such as in patient rooms 42, patient bathrooms 44, along a hallway 46, and at a nurses station 48. The fluid dispensers 10 located in the patient rooms 42, hallway 46, and nurses station 48 can be sanitizer dispensers, while the fluid dispensers 10 located in the patient bathrooms 44 can be soap dispensers. Each time a patient uses one of the fluid dispensers 10, the fluid dispenser 10 can report the patient's usage to the central monitoring system 34 and include such information as patient identification (e.g., from the RFID tag 36), fluid dispenser identification number, time of usage, fluid type, etc. It is to be appreciated that fluid dispensers can be provided in any of a variety of suitable additional or alternative locations in a health care facility for encouraging patient hand-hygiene.

The health care facility 40 can also include other hygiene devices (e.g., hand-hygiene devices and/or general purpose hygiene devices) that are in communication with, and monitored by, the remote monitoring station 34. For example, sanitizing glove dispensers (not shown) can be provided throughout the health care facility for patient use (e.g., for handling hazardous waste). Each time a patient removes a glove from the glove dispenser, the glove dispenser can report the event to the central monitoring system 34 (e.g., for logging) including such information as patient identification (e.g., from the RFID tag 36), glove dispenser identification number, time of usage, glove type, quantity of gloves, etc. In another example, sharps containers (not shown) can be provided throughout the health care facility 40 for disposal of sharps devices (e.g., contaminated needles). Each time a patient disposes of a sharps device into the sharps container, the sharps containers can report the event to the central monitoring system 34 (e.g., for logging thereto) including such information as patient identification (e.g., from the RFID tag 36), sharps container identification number, time of disposal, sharps type disposed of, etc.

The health care facility 40 can further include non-sanitizing devices that are in communication with, and monitored by, the remote monitoring station 34, such as, for example, a toilet, a sink, a door, or a vending machine. Each of these non-sanitizing devices can be equipped with an RFID reader or other device for recognizing an approaching patient. When the patient interacts with the non-sanitizing device, the non-sanitizing device can report the interaction to the remote monitoring station 34.

Referring again to FIG. 3, the central monitoring system 34 can include a processor 50 that is associated with a patient profile database 52. The patient profile database 52 can include patient profiles 54a, 54b, 54c, . . . 54n for different patients being served by the health care facility. As illustrated in FIG. 3, each of the patient profiles 54a, 54b, 54c, . . . 54n can include a hand-hygiene compliance (HHC) protocol 56a, 56b, 56c, . . . 56n for the patient that is associated with the patient profile. Each HHC protocol 56a, 56b, 56c, . . . 56n can define suggested hand-hygiene related activities for each patient such as, for example, sanitizing hands with sanitizer, washing hands, and/or wearing protective gloves. The HHC protocol 56a, 56b, 56c, . . . 56n can also define certain parameters for the hand-hygiene related activities, such as frequency of hand sanitizing/washing and the type of fluid that needs to be used for sanitizing.

Each of the suggested hand-hygiene activities and parameters of the HHC protocols 56a, 56b, 56c, . . . 56n can either be scheduled or behavior-based. The scheduled activities and parameters can be prescribed according to various health related factors of the patient, such as, for example, health condition, age, capability, condition, and/or reason(s) for previous admittance to the health care facility. For example, patients with a health condition that causes them to be susceptible to infection (e.g., auto-immune deficiencies, MRSA, etc.) may be required to sanitize their hands more frequently than other patients and/or use different (stronger) fluids than other patients. Older patients may be required to sanitize more frequently than younger patients. Bed ridden (e.g., comatose) patients may not be required to sanitize hands at all.

As illustrated in FIG. 3, each of the patient profiles 54a, 54b, 54c, . . . 54n can include personal data fields 60a, 60b, 60c, . . . 60n where the health related factors for each patient can be stored. It is also to be appreciated that the patient's health related factors can be collected from any of a variety of sources, such as, for example, directly from the patient (e.g., upon admittance of the patient to the health care facility), from a physician who diagnosed the patient prior to the patient entering the health care facility, from a physician who diagnosed the patient during the patient's stay at the health care facility, from a questionnaire presented to the patient upon checking into the health care facility and/or from the patient's electronic medical record (EMR).

In one embodiment, as illustrated in FIG. 3, the central monitoring system 34 can include an HHC generation module 58 that is configured to generate the scheduled activities and parameters for each patient's HHC protocol based upon each patient's health related factors. In such an embodiment, the relevant health related factors for each patient can be collected and provided to the HHC generation module 58. The HHC generation module 58 can then generate a personalized HHC protocol (e.g., 56a, 56b, 56c, . . . 56n) for the patient based upon the patient's health related factors and can provide the personalized HHC protocol to the patient's patient profile (e.g., 54a, 54b, 54c, . . . 54n). In another embodiment, the patient's personalized HHC protocol can be manually generated by health care facility personnel and entered into the patient's patient profile (e.g., 54a, 54b, 54c, . . . 54n) via an input device (e.g., a keyboard). It is to be appreciated that although the HHC generation module 58 is shown to be provided onboard the central monitoring system 34, the HHC generation module 58 can be provided as part of any of a variety of other suitable computer systems, such as in an online database maintained by a third party.

The behavior-based activities and parameters for each patient's HHC protocol can be generated automatically and in substantially real-time based upon the patient's hygiene related conduct within the health care facility. For example, each time the patient uses the restroom, opens a door, or operates a vending machine, the HHC protocol for the patient can be updated to include a suggested hand-hygiene activity, such as washing hands at a soap dispenser, within a predefined time (e.g., 30 seconds). As illustrated in FIG. 3, each of the patient profiles 54a, 54b, 54c, . . . 54n can include non-hygiene activity logs 62a, 62b, 62c, . . . 62n where the patient's non-sanitizing activity is logged.

The personalized HHC protocols for each patient can include any of a variety of rules that can determine whether the patient has complied with a personalized HHC protocol. One example rule can be a window of time (e.g., +/−30 minutes) in which the patient can sanitize his/her hands to meet a particular suggested activity. Other rules can override scheduled activities. For example, when a patient goes to sleep, the hand-hygiene activities that are scheduled during the patient's sleep can be overridden to account for the patient's inactivity/inability to properly sanitize his/her hands.

As the patient moves through the health care facility, the central monitoring system 34 can collect the patient's hand-hygiene activity from the hand-hygiene devices (e.g., fluid dispensers 10) that the patient operates and can log them to the patient's hygiene activity log (see 64a, 64b, 64c, . . . 64n). In one embodiment, the fluid dispenser 10 can store the hand-hygiene activity to an on-board data store (e.g., a memory module) (not shown). Periodically (e.g., once a day), the central monitoring system 34 can retrieve the hygiene-related activity from the data store of the fluid dispenser 10. In another embodiment, the fluid dispenser 10 can transmit the patients' hand-hygiene activity in substantially real time to the central monitoring system 34.

The central monitoring system 34 can compare the patient's hand-hygiene activity with the patient's personalized HHC protocol. Each time the patient does not comply with a suggested activity/parameter (e.g., scheduled or behavior-based) from his/her personalized HHC, a violation record can be logged to the patient's patient profile (e.g., 54a, 54b, 54c, . . . 54n). The violation record can indicate the nature of the violation, such as, for example, hand sanitizing not performed, wrong fluid used, and/or protective gloves not used. The violation record can also include other pertinent information such as, the date and time of the violation and/or the fluid dispensers (e.g., 10) that were in proximity to the patient when the violation occurred. As illustrated in FIG. 3, each of the patient profiles 54a, 54b, 54c, . . . 54n can include a violation log 66a, 66b, 66c, . . . 66n where the patient's violations are logged.

In one embodiment, the violation record(s) recorded to the patient's profile can be provided to the patient and/or the patient's care giver (e.g., as printout or on a computer screen) to encourage the patient to improve his/her hand-hygiene while staying at the health care facility. In another embodiment, the violation record(s) recorded to the patient's profile can be recorded to the patient's EMR. In such an embodiment, the violation record(s) can be used to limit the health care facility's liability if the patient were to contract an infection (e.g., MRSA) while admitted at the health care facility. In another embodiment, the violation log can be used to create a work order for the patient that includes the violation log and identifies each of the suggested hand-hygiene related activities from the HHC protocol with which the patient complied. Identifying violations in this manner can help alleviate proliferation of healthcare-associated infections (HAIs) in the healthcare facility.

In one embodiment, when a violation record is recorded to the patient's patient profile, the fluid dispenser(s) 10 closest to the patient can alert the patient that he/she is in violation of the HHC protocol such as, for example, with a visible and/or audible alert at the fluid dispenser(s) 10 and/or on the RFID tag 36. The most proximate fluid dispenser(s) 10 to the patient can be determined via a received signal strength indicator (RSSI) algorithm or any of a variety of suitable alternative methods. If the patient then takes corrective action by actuating one of the fluid dispensers 10 within a predefined amount of time, the violation record can be deleted from the patient's patient profile (e.g., 54a, 54b, 54c, . . . 54n).

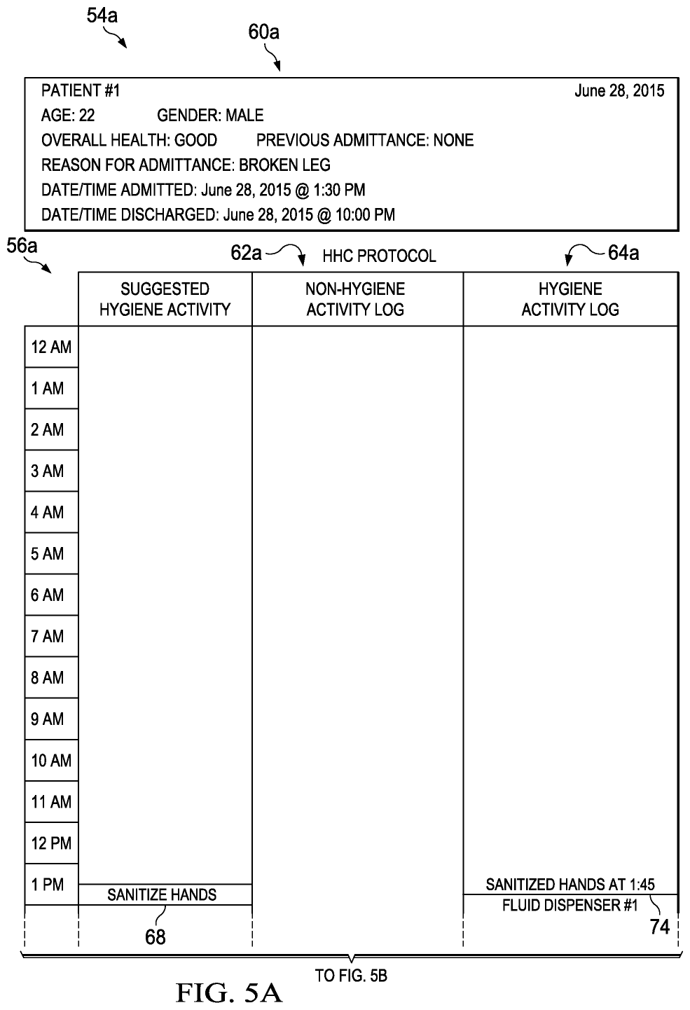

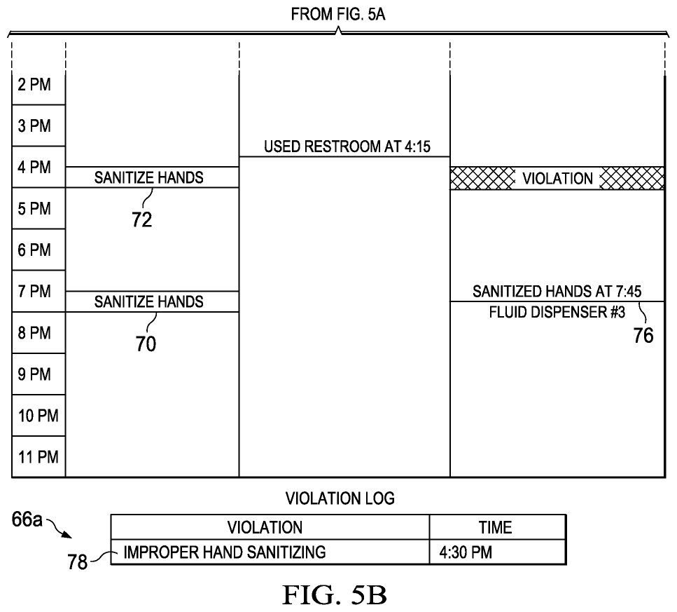

Referring now to FIGS. 5A, 5B, 6A, 6B, 7A, and 7B, the patient profiles 54a, 54b, 54c of FIG. 3 are shown to be populated with 24 hours of example historical data for a first patient (Patient #1), a second patient (Patient #2), and a third patient (Patient #3), respectively. Referring now to the patient profile 54a of FIGS. 5A and 5B, Patient #1 is a 22 year old male who is in good health and was admitted to the hospital at 1:30 P.M. on Jun. 28, 2015, for a broken leg and discharged at 10:00 P.M. the same day (see personal data field 60a). The personalized HHC protocol 56a includes scheduled hygiene activities 68, 70 that suggest that Patient #1 sanitize his hands between 1:30 P.M. and 2:00 P.M. and between 7:30 P.M. and 8:00 P.M. (e.g., every 6 hours with a 30 minute window). The non-sanitizing activity log 62a indicates that Patient #1 used the restroom at 4:15 P.M. The personalized HHC protocol 56a, therefore, also includes a behavior-based scheduled hygiene activity 72 that suggests that Patient #1 sanitize his hands between 4:15 P.M. and 4:30 P.M. The hygiene log indicates that Patient #1 sanitized his hands at 1:45 P.M. at fluid dispenser #1 (see 74) and at 7:45 at fluid dispenser #3 (see 76). However, Patient #1 did not sanitize his hands appropriately after using the restroom at 4:15 P.M. As a result, a violation 78 is added to the violation log 66a for Patient #1.

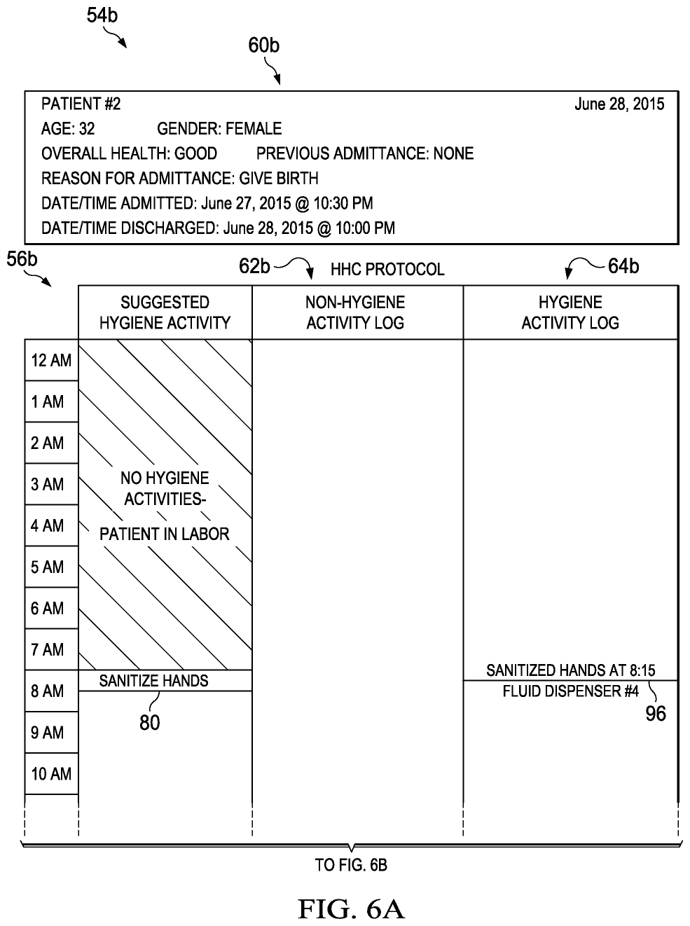

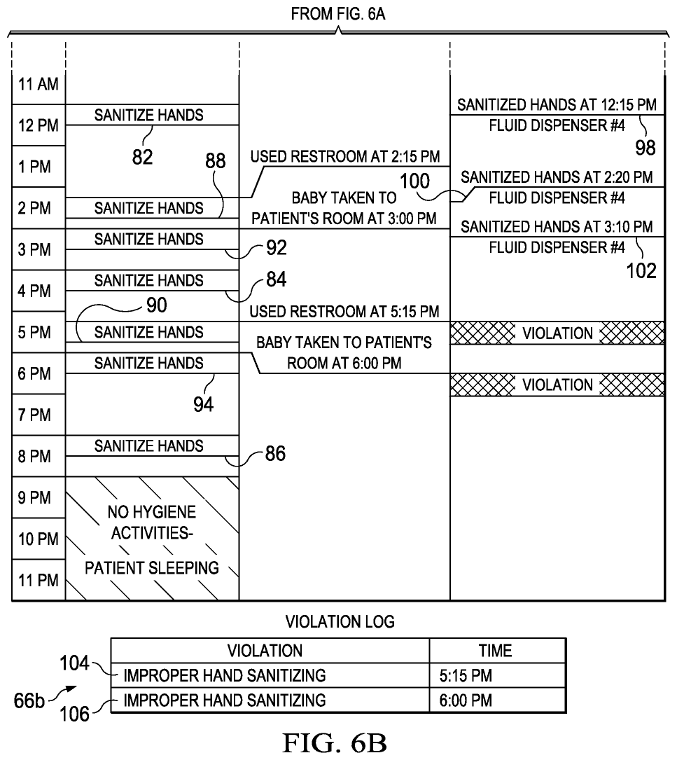

Referring now to the patient profile 54b of FIGS. 6A and 6B, Patient #2 is a 32 year old female in good health who was admitted to the hospital to give birth at 10:30 P.M. on Jun. 27, 2015, and was discharged at 10:00 P.M. on June 28th (see personal data field 60b). The example historical data for the patient profile 54b is for Jun. 28, 2015. The personalized HHC protocol 56b shows that Patient #2 was in labor until 8:00 A.M., and thus no hygiene activities were scheduled. After 8:00 A.M., the HHC protocol scheduled hygiene activities 80, 82, 84, 86 suggest that Patient #2 sanitize her hands between 8:00 A.M. and 8:30 A.M., between 12:00 P.M. and 12:30 P.M., between 4:00 P.M. and 4:30 P.M., and between 8:00 P.M. and 8:30 P.M. (e.g., every 4 hours with a 30 minute window). After 9:00 P.M., the personalized HHC protocol 56b shows that Patient #2 was asleep, and thus no hygiene activities were scheduled. The non-sanitizing activity log 62b indicates that Patient #2 used the restroom at 2:15 P.M. and 5:15 P.M. As a result, the personalized HHC protocol 56b includes behavior-based scheduled hygiene activities 88, 90 that suggest that Patient #2 sanitize her hands between 2:15 P.M. and 2:30 P.M. and between 5:15 P.M. and 5:30 P.M., respectively. The non-sanitizing activity log 62b also indicates that the baby was brought to Patient #2 at 3:00 P.M. and 6:00 P.M. As a result, the personalized HHC protocol 56b includes behavior-based scheduled hygiene activities 92, 94 that suggest that Patient #2 should sanitize her hands between 3:00 P.M. and 3:30 P.M. and between 6:00 P.M. and 6:30 P.M., respectively. The hygiene activity log 64b indicates that Patient #2 sanitized her hands at 8:15 A.M. at fluid dispenser #4 (see 96), at 12:15 P.M. at fluid dispenser #4 (see 98), 2:20 P.M. at fluid dispenser #4 (see 100), and at 3:10 P.M. at fluid dispenser #4 (see 102). However, Patient #2 did not sanitize her hands appropriately after using the restroom at 5:15 P.M. or when her baby was brought to her at 6:00 P.M. As a result, violations 104, 106 are added to the violation log 66b for Patient #2.

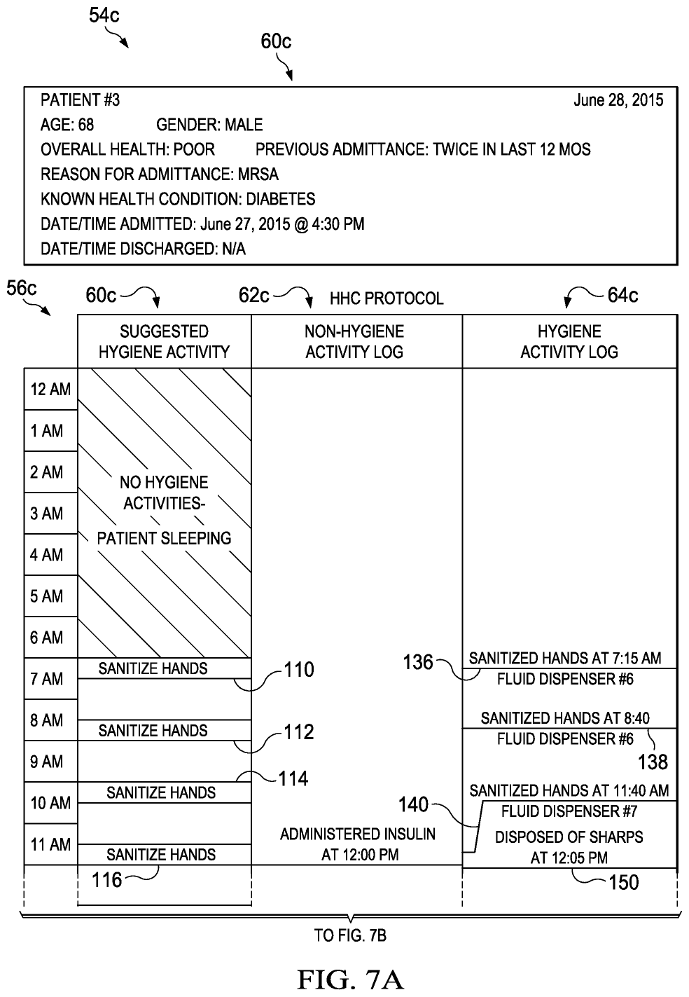

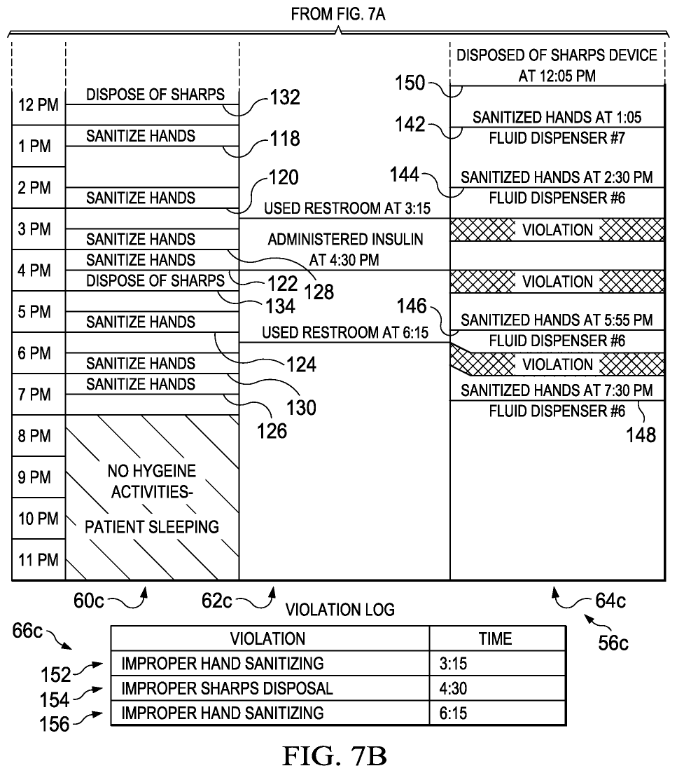

Referring now to the patient profile 54c of FIGS. 7A and 7B, Patient #3 is a 68 year old diabetic male, who is in poor health and has been admitted to the health care facility twice in the last 12 months, has MRSA, and was admitted to the hospital at 4:30 P.M. on Jun. 27, 2015 (see personal data field 60c). The example historical data for the patient profile 54c is for Jun. 28, 2015. The personalized HHC protocol 56c shows that Patient #3 was asleep until 7:00 A.M., and thus no hygiene activities were scheduled. After 7:00 A.M., the HHC protocol scheduled hygiene activities 110, 112, 114, 116, 118, 120, 122, 124, 126 suggest that Patient #3 sanitize his hands between 7:00 A.M. and 7:30 A.M., between 8:30 A.M. and 9:00 A.M., between 10:00 A.M. and 10:30 A.M., between 11:30 A.M. and 12:00 P.M., between 1:00 P.M. and 1:30 P.M., between 2:30 P.M. and 3:00 P.M., between 4:00 P.M. and 4:30 P.M., between 5:30 P.M. and 6:00 P.M., and between 7:00 P.M. and 7:30 P.M. (e.g., every 1.5 hours with a 30 minute window). After 8:00 P.M., the personalized HHC protocol 56c shows that Patient #3 was asleep and thus no hygiene activities were scheduled. The non-sanitizing activity log 62b indicates that Patient #2 used the restroom at 3:15 P.M. and 6:15 P.M. As a result, the personalized HHC protocol 56c includes behavior-based scheduled hygiene activities 128, 130 that suggest that Patient #3 sanitize his hands between 3:15 P.M. and 3:30 P.M. and between 6:15 P.M. and 6:30 P.M., respectively. The non-sanitizing activity log 62c also indicates that Patient #3 self-administered insulin at 12:00 P.M. and 4:30 P.M. As a result, the personalized HHC protocol 56c includes behavior-based scheduled hygiene activities 132, 134 that suggest that Patient #3 should dispose of the needles in the sharps container between 12:00 P.M. and 12:30 P.M. and between 4:30 P.M. and 5:00 P.M., respectively. The hygiene activity log 64c indicates that Patient #3 sanitized his hands at 7:15 A.M. at fluid dispenser #6 (see 136), at 8:40 A.M. at fluid dispenser #6 (see 138), at 11:40 A.M. at fluid dispenser #7 (see 140), at 1:05 P.M. at fluid dispenser #7 (see 142), at 2:30 P.M. at fluid dispenser #6 (see 144), at 5:55 P.M. at fluid dispenser #6 (see 146), and at 7:30 P.M. at fluid dispenser #6 (see 148). The hygiene activity log 64c also indicates that the patient disposed of the sharps device at 12:05 P.M. (see 150). However, Patient #3 did not sanitize his hands appropriately after using the restroom at 3:15 P.M. and did not dispose of the needle in the sharps container after self-administering insulin at 4:30 P.M. As a result, violations 152, 154, 156 are added to the violation log 66c for Patient #3.

The fluid dispenser 10, the RFID tag 36, and the other devices in communication with the central monitoring system 34 (collectively “the communication devices”) can include a processor (not shown) that can be any suitable type of processing unit, for example a general purpose central processing unit (CPU), a reduced instruction set computer (RISC), a processor that has a pipeline or multiple processing capability including having multiple cores, a complex instruction set computer (CISC), a digital signal processor (DSP), an application specific integrated circuit (ASIC), a programmable logic device (PLD), and a field programmable gate array (FPGA), among others. The communication devices can include or otherwise facilitate various other computing resources such as, for example, distributed computing devices, cloud computing resources, and virtual computing resources in general.

The communication devices can include network and communication interfaces that allow communication with other devices across a network. The network and communication interfaces can be an Ethernet interface, a radio interface, a telephony interface, a Universal Serial Bus (USB) interface, or any other suitable communications interface. Example communication interfaces can include wired data transmission links such as Ethernet and TCP/IP, as well as PSTN communications links such as T1s (or better), integrated services digital network (ISDN), Digital Subscriber Line (DSL), or dialup modems that implement, for example, the point-to-point protocol (PPP). The communication interface can include wireless protocols for interfacing with private or public networks. For example, the network and communication interfaces and protocols can include interfaces for communicating with private wireless networks such as a WiFi network, one of the IEEE 802.11x family of networks, or another suitable wireless network. The network and communication interfaces can include interfaces and protocols for communicating with public wireless networks, using for example wireless protocols used by cellular network providers, including Code Division Multiple Access (CDMA) and Global System for Mobile Communications (GSM). The communication devices can use network and communication interfaces to communicate with hardware modules such as a database or data store, or one or more servers or other networked computing resources. Data can be encrypted or protected from unauthorized access.

In various configurations, the communication devices, or other computing device, can include a system bus for interconnecting various components, and/or can be integrated into one or more chips such as a programmable logic device or application specific integrated circuit (ASIC). The system bus can include a memory controller, a local bus, or a peripheral bus for supporting input and output devices, or communication interfaces. Example input and output devices include keyboards, keypads, gesture or graphical input devices, motion input devices, touchscreen interfaces, displays, audio units, voice recognition units, vibratory devices, computer mice, and any other suitable user interface.

The processor can include nonvolatile memory for storing computer-readable instructions, data, data structures, program modules, code, microcode, and other software components for storing the computer-readable instructions in non-transitory computer-readable mediums in connection with the other hardware components for carrying out the methodologies described herein. Software components can include source code, compiled code, interpreted code, executable code, static code, dynamic code, encrypted code, or any other suitable type of code or computer instructions implemented using any suitable high-level, low-level, object-oriented, visual, compiled, or interpreted programming language.

It is to be appreciated that any of a variety of suitable alternative hygiene protocols can be provided for any of a variety of hygiene related activities. The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent devices by those of ordinary skill in the art. Rather it is hereby intended the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is claimed is:

1. A method for creating personalized hand hygiene compliance protocols and for tracking hand hygiene compliance comprising:
providing a plurality of dispensers;
wherein the dispensers contain one of soap or sanitizer;
retrieving patient profile information from a patient profile database that relates to a patient;
creating a personalized hand-hygiene compliance protocol for the patient as a function of the patient profile information;
wherein the hand-hygiene compliance protocol includes at least two conditions that require the patient to wash her hands with soap or sanitizer;
monitoring the plurality of dispensers;
determining if a dispense from one of the plurality of dispensers was caused by the patient;
logging the dispense from one of the plurality of dispensers; and
associating the dispense to the patient if the dispense was caused by the patient.

2. The method of claim 1 further comprising providing a badge for the patient, wherein the badge comprises information that is used to identify the patient.

3. The method of claim 2 wherein the badge comprises an RFID.

4. The method of claim 1 further comprising providing a report that identifies violations of the personalized hand-hygiene compliance protocol by the patient.

5. The method of claim 1 further comprising providing a report that identifies compliance of the personalized hand-hygiene compliance protocol by the patient.

6. The method of claim 1 further comprising monitoring use of a restroom by the patient.

7. The method of claim 1 further comprising monitoring use of a door by the patient.

8. The method of claim 1 wherein the personalized hand-hygiene compliance protocol includes at least one requirement for the patient to wash her hands with soap and at least one requirement for the patient to sanitizer her hands with sanitizer.

9. The method of claim 1 wherein the non-sanitizing device is one or more of a toilet, a urinal, a drinking fountain, and a sink and the sanitizing device is a hand liquid dispenser.

10. The method of claim 1 wherein determining if a dispense from one of the plurality of dispensers was caused by the patient comprises:
receiving biometric information from the patient at the dispenser.

11. The method of claim 1 further comprises activating an indicator proximate a dispenser to indicate to the patient that she needs to wash or sanitizer her hands.

12. The method of claim 11 wherein activating an indicator comprises illuminating a light.

13. The method of claim 1 wherein the patient profile comprises one or more of age, capability, condition and demographic.

14. The method of claim 1 further comprising determining whether the patient is sleeping and wherein violations that occur while the person is sleeping are not reported as violations.

15. A method for developing a personalized hand-hygiene compliance protocol and tracking compliance thereof comprising: providing a plurality of dispensers;
providing a plurality of hand-hygiene dispensers;
wherein the hand-hygiene dispensers contain one of soap or sanitizer;
obtaining patient profile information that relates to a patient;
wherein the patient profile includes a reason for admittance to the hospital;
creating a personalized hand-hygiene compliance protocol for the patient as a function of the patient profile information;
wherein the personalized hand-hygiene compliance protocol includes at least two conditions that require the patient to wash her hands with soap or sanitizer;
monitoring the plurality of dispensers;
determining if a dispense from one of the plurality of dispensers was caused by the patient;
logging the dispense from one of the plurality of dispensers; and
associating the dispense to the patient if the dispense was caused by the patient.

16. The method of claim 15 wherein the patient profile information comprises a health condition.

17. The method of claim 15 wherein the patient profile information comprises a health condition.

18. A method for creating personalized hand hygiene compliance protocols and for tracking hand hygiene compliance comprising:
providing a plurality of dispensers;
wherein the dispensers contain one of soap or sanitizer;
using an input device to input patient profile information to a central computer;
preparation of a custom hand-hygiene compliance protocol for the patient as a function of the patient profile information;
wherein the hand-hygiene compliance protocol includes at least two conditions that require the patient to wash her hands with soap or sanitizer;
transmitting a signal indicative of a dispense event when a dispenser dispensing a dose of fluid;
logging the dispense event from the one of the plurality of dispensers;
associating the dispense event to the patient if the dispense was caused by the patient; and
providing a compliance report indicative of whether the patient has complied with the custom hand-hygiene compliance protocol.

19. The method of claim 18 wherein the patient profile information is input by a doctor.

20. The method of claim 18 wherein the patient profile information is stored in a patient profile database.
